(19) United States
(12) Patent Application Publication
(10) Pub. No.: US 2018/0142036 A1
(43) Pub. Date: May 24, 2018

(54) COMPOSITION ENRICHED IN ANTI-A AND/OR ANTI-B POLYCLONAL IMMUNOGLOBULINS FOR USE IN THE TREATMENT OF AUTOIMMUNE DISEASES OR POLYCYTHEMIA

(71) Applicant: Laboratoire Francais du Fractionnement et des Biotechnologies, Les Ulis (FR)

(72) Inventor: Abdessatar Sami CHTOUROU, Elancourt (FR)

(73) Assignee: Laboratoire Francais du Fractionnement et des Biotechnologies, Les Ulis (FR)

(21) Appl. No.: 15/570,511

(22) PCT Filed: May 6, 2016
(86) PCT No.: PCT/EP2016/060157

§ 371 Date: Oct. 30, 2017

(30) Foreign Application Priority Data
May 7, 2015 (FR) 1554117

(51) Int. Cl. C07K 16/34; A61K 38/36; C07K 16/06; A61P 37/00; A61P 35/00; A61P 7/02

(52) U.S. Cl. CPC C07K 16/34; A61K 38/36; C07K 16/065; A61K 2039/505; A61P 35/00; A61P 7/02; A61P 37/00

(57) ABSTRACT

The invention relates to a composition strongly enriched with anti-A and/or anti-B polyclonal immunoglobulins, comprising polyclonal human immunoglobulins, characterised in that at least 80 wt.-% of the polyclonal human immunoglobulins present in the composition are anti-A or anti-B polyclonal human immunoglobulins, for use as a drug, particularly in the treatment of polycythemia and/or autoimmune diseases, and in particular peripheral autoimmune thrombocytopenia.

### FIELD OF THE INVENTION

The present invention falls within the field of therapeutic polyclonal immunoglobulin concentrates. It concerns a composition that is highly enriched in anti-A and/or anti-B polyclonal immunoglobulins, for use in the treatment of autoimmune diseases (in particular idiopathic thrombocytopenic purpura), and/or in the treatment of polycythemia.

### PRIOR ART

Human normal immunoglobulins (human polyvalent immunoglobulins purified from plasma pooled from at least 1000 donors) are used to treat a growing number of pathologies. They are used in particular as replacement therapies in primary immunodeficiencies (congenital deficiencies) or secondary immunodeficiencies (chronic lymphocytic leukemia, myeloma, post-bone marrow transplant infections, recurrent bacterial infections in HIV-infected children) with an antibody production defect. They are also used as an immunomodulatory treatment in various autoimmune pathologies such as idiopathic thrombocytopenic purpura (ITP), Birdshot's retinochoroiditis, Guillain-Barré syndrome, multifocal motor neuropathy (MMN), chronic inflammatory demyelinating polyneuropathies (CIDP), or Kawasaki's disease.

This growing use requires an increasingly greater supply of human normal immunoglobulins. This leads to the use of increasingly large donor pools. A significant proportion of donors are blood group O, and consequently their plasma contains anti-A and anti-B immunoglobulins, directed against antigens of blood groups A and B. Furthermore, blood group A donors generally have anti-B immunoglobulins and blood group B donors generally have anti-A immunoglobulins. Only blood group AB donors do not have anti-A and anti-B immunoglobulins, but these donors are rare.

However, when the proportions of anti-A and anti-B immunoglobulins in human normal immunoglobulins are too high, they are likely to cause accidental hemolysis, which is potentially severe, in treated patients carrying A and/or B antigens.

Consequently, health authorities require human normal immunoglobulins to be tested with regard to blood groups A and B red blood cell agglutination activity, and set limits for this activity. Thus, for a long time, according to the European Pharmacopoeia, human normal immunoglobulins for intravenous administration (IVIG) must not show A and B red blood cell agglutination at a 1:64 dilution of a solution whose initial concentration is 50 g/L (5%), in a specific direct agglutination test corresponding to that developed by Thorpe and colleagues (Thorpe et al. Biologicals. 2005 June; 33(2): 111-6; Thorpe et al. Vox Sang. 2009 August; 97(2):160-8; Thorpe et al. Pharmeur Bio Sci Notes. 2010 April; 2010(1):39-50; European Pharmacopoeia, Chapter 2.6.20 as revised by Supplement 7.2 of January 2011). With the aim of avoiding accidental hemolysis without reducing the donor pools able to be used, plasma fractionators have developed methods for eliminating anti-A and anti-B immunoglobulins from their human normal immunoglobulin concentrates.

For example, WO01/27623 and WO2007/077365 describe the use of various affinity chromatography supports that specifically bind to anti-A and anti-B immunoglobulins in order to eliminate anti-A and anti-B immunoglobulins from biological compositions, in particular from human normal immunoglobulin concentrates. The unadsorbed fraction not comprising anti-A and anti-B immunoglobulins is collected by percolation, for subsequent treatment and packaging. The anti-A anti-B affinity chromatography column is then regenerated by a double washing: a first acid washing, followed by a second base washing. Heretofore, the two products obtained from each of the two washings have been removed. The anti-A and anti-B immunoglobulin fraction adsorbed during the affinity chromatography step thus corresponds to date to a lost fraction.

### SUMMARY OF THE INVENTION

However, in the context of the present invention, the inventors have discovered that the currently removed fraction, which is highly enriched in anti-A and anti-B immunoglobulins, could be used in the treatment of autoimmune diseases (in particular idiopathic thrombocytopenic purpura (ITP)), and/or in the treatment of polycythemia.

A hypothesis to explain the efficacy of purified anti-A and/or anti-B immunoglobulin compositions in the treatment of autoimmune diseases and in particular of ITP is that these anti-A and/or anti-B immunoglobulins, administered to a blood group A, B or AB patient with an autoimmune disease, will compete with endogenous phenomena. Anti-A and/or anti-B immunoglobulins would make it possible to induce destruction of the patient's erythrocytes by a phenomenon of phagocytosis of an erythrocyte fraction and would thus indirectly protect platelets from destruction by macrophages. Indeed, macrophage Fc receptors would be saturated directly by anti-A and/or anti-B immunoglobulin-coated erythrocytes, thereby limiting and/or preventing platelet destruction.

A second possible mechanism is based on consumption of complement proteins by anti-A and/or anti-B immunoglobulin-coated erythrocytes, reducing the complement-dependent activity of pathogenic autoantibodies. This mechanism applies to all autoimmune pathologies for which the role of the complement is harmful to the patient, such as, for example, neuromyelitis optica (anti-AQP4) and multiple sclerosis (anti-myelin/axon).

A third possible mechanism is an anti-inflammatory action of anti-A and/or anti-B immunoglobulin-coated erythrocytes, due to inhibition induced during their phagocytosis of proinflammatory cytokine secretion by activated macrophages. This mechanism applies to all autoimmune pathologies for which effector cells expressing Fc receptors, in particular CD16, are activated and maintain a pathogenic inflammatory state in the patient, which is observed in a large number of autoimmune pathologies.

An immunomodulation phenomenon is also possible. Indeed, a fourth possible mechanism is based on the immunomodulatory action of anti-A and/or anti-B immunoglobulin-coated erythrocytes which, by limiting cell destruction, allows the induction of a negative signal and thus a decrease in autoantibody secretion by cross-linking between BCR and FcgRIIb of the B cell via the autoantibody and the antigen expressed on the target cell surface. This mechanism applies to all autoimmune pathologies for which a pathogenic autoantibody is shown, such as ITP, insulin autoimmune syndrome (IAS) or Hirata disease (anti-insulin), neuromyelitis optica (anti-AQP4), Grave's disease, pemphigus, multiple sclerosis (anti-myelin/axon), Sydenham's chorea (anti-neuronal protein), Sjögren's syndrome, pemphigus vulgaris (anti-desmoglein 3).

A fifth mechanism is induction of anti-inflammatory cytokines such as IL1-RA by immune system cells, epithelial cells and adipocytes induced by anti-A and/or anti-B immunoglobulin-coated erythrocytes. This mechanism applies in particular in inflammatory pathologies such as rheumatoid arthritis or for example Muckle-Wells syndrome and Schnitzler syndrome.

In the case of polycythemia, anti-A and/or anti-B immunoglobulins will bind directly to the erythrocytes of blood group A, B or AB patients and will thus induce lysis of surplus erythrocytes.

In a first aspect, the present invention thus relates to a composition comprising human polyclonal immunoglobulins, characterized in that at least 80% by weight of human polyclonal immunoglobulins present in the composition are human anti-A and/or anti-B polyclonal immunoglobulins, for use as a medicament.

The invention also relates to a composition comprising human polyclonal immunoglobulins, characterized in that at least 80% by weight of human polyclonal immunoglobulins present in the composition are human anti-A and/or anti-B polyclonal immunoglobulins, for use as a medicament in the treatment of autoimmune diseases (in particular idiopathic thrombocytopenic purpura (ITP)) and/or in the treatment of polycythemia.

Advantageously, human polyclonal immunoglobulins represent at least 85% by weight of the total proteins of the composition.

The invention also relates to the use of a composition comprising human polyclonal immunoglobulins, characterized in that at least 80% by weight of human polyclonal immunoglobulins present in the composition are human anti-A and/or anti-B polyclonal immunoglobulins, for the preparation of a medicament, in particular intended for the treatment of autoimmune diseases (in particular idiopathic thrombocytopenic purpura (ITP)) and/or for the treatment of polycythemia.

The invention also relates to a method for treating autoimmune diseases (in particular idiopathic thrombocytopenic purpura (ITP)) and/or polycythemia in a subject in need thereof, comprising administering to said subject an effective amount of a composition comprising human polyclonal immunoglobulins, characterized in that at least 80% by weight of human polyclonal immunoglobulins present in the composition are human anti-A and/or anti-B polyclonal immunoglobulins.

Advantageously, these compositions are intended for blood group A, B or AB patients.

In still another advantageous embodiment, the composition comprises both human anti-A polyclonal immunoglobulins and human anti-B polyclonal immunoglobulins, and the weight ratio of human anti-A polyclonal immunoglobulins to human anti-B polyclonal immunoglobulins (anti-A:anti-B) is comprised between 1:10 and 10:1. In this case, the composition is advantageously intended for blood group A, B or AB patients.

### DESCRIPTION OF THE FIGURES

FIG. 1. Diagram of the murine model used to test the therapeutic efficacy of anti-A and/or anti-B polyclonal immunoglobulins in the treatment of idiopathic thrombocytopenic purpura (ITP). The arrows above the timeline correspond to blood collections. The short, solid arrows below the timeline correspond to injections of anti-CD41 antibody (1 μg/20 g weight for each mouse, intraperitoneally). The long, dotted arrows beneath the timeline correspond to injection of human AB+ erythrocytes (400 μL, intravenous), or to injection of IVIG (2 g/kg, intraperitoneally) or anti-A/anti-B (7.5 mg/kg, intraperitoneally).

FIG. 2. Graphical representation of the results obtained, expressed as a percentage of day 1, on the murine model used to test the therapeutic efficacy of anti-A and/or anti-B polyclonal immunoglobulins in the treatment of idiopathic thrombocytopenic purpura (ITP). Gr1: group 1; Gr2: group 2; Gr3: group 3; Gr4: group 4; Gr5: group 5.

### DETAILED DESCRIPTION OF THE INVENTION

In the context of the present invention, the inventors have discovered that the fraction that is currently eliminated, which is highly enriched in anti-A and anti-B immunoglobulins, could be used in the treatment of autoimmune diseases (in particular idiopathic thrombocytopenic purpura (ITP)), and/or in the treatment of polycythemia.

A hypothesis to explain the efficacy of purified anti-A and/or anti-B immunoglobulin compositions in the treatment of autoimmune diseases and in particular of ITP is that these anti-A and/or anti-B immunoglobulins, administered to a blood group A, B or AB patient with an autoimmune disease, will compete with endogenous phenomena. Anti-A and/or anti-B immunoglobulins would make it possible to induce destruction of the patient's erythrocytes and thus would, indirectly, protect platelets from destruction by macrophages. Indeed, macrophage Fc receptors would be saturated directly by anti-A and/or anti-B immunoglobulin-coated erythrocytes, thus preventing platelet destruction. A second possible mechanism is based on a consumption of complement proteins by anti-A and/or anti-B immunoglobulin-coated erythrocytes, reducing the complement-dependent activity of pathogenic autoantibodies. This mechanism applies to all autoimmune pathologies for which the role of the complement is harmful to the patient, such as, for example, neuromyelitis optica (anti-AQP4) and multiple sclerosis (anti-myelin/axon).

A third possible mechanism is an anti-inflammatory action of anti-A and/or anti-B immunoglobulin-coated erythrocytes, due to inhibition induced during their phagocytosis of secretion of proinflammatory cytokines by activated macrophages. This mechanism applies to all autoimmune pathologies for which effector cells expressing Fc receptors, in particular CD16, are activated and maintain a pathogenic inflammatory state in the patient, which is observed in a large number of autoimmune pathologies.

An immunomodulation phenomenon is also possible. Indeed, a fourth possible mechanism is based on the immunomodulatory action of anti-A and/or anti-B immunoglobulin-coated erythrocytes which, by limiting cell destruction, allows the induction of a negative signal and thus a decrease in autoantibody secretion by cross-linking between BCR and FcgRIIb of the B cell via the autoantibody and the antigen expressed on the target cell surface. This mechanism applies to all autoimmune pathologies for which a pathogenic autoantibody is shown, such as ITP, insulin autoimmune syndrome (IAS) or Hirata disease (anti-insulin), neuromyelitis optica (anti-AQP4), Grave's disease, pemphigus, multiple sclerosis (anti-myelin/axon), Sydenham's chorea (anti-neuronal protein), Sjögren's syndrome, pemphigus vulgaris (anti-desmoglein 3).

A fifth mechanism is the induction of anti-inflammatory cytokines such as IL1-RA by immune system cells, epithelial cells and adipocytes induced by anti-A and/or anti-B immunoglobulin-coated erythrocytes. This mechanism applies in particular in inflammatory pathologies such as rheumatoid arthritis or for example Muckle-Wells syndrome and Schnitzler syndrome.

In the case of polycythemia, anti-A and/or anti-B immunoglobulins will bind directly to the erythrocytes of blood group A, B or AB patients thereby inducing lysis of surplus erythrocytes.

### Definitions

By “antibody” or “immunoglobulin” is meant a molecule comprising at least one binding domain for a given antigen and a constant domain comprising an Fc fragment capable of binding to Fc receptors (FcR).

By “human polyclonal immunoglobulins” is meant a composition of human immunoglobulins directed against numerous distinct antigens, and comprising, for each recognized antigen, multiple distinct immunoglobulins capable of recognizing said antigen, generally at several distinct epitopes. Such human polyclonal immunoglobulins are generally purified from plasma from one donor or, preferably, from several donors (called a donor pool). Therapeutic human normal immunoglobulins are thus purified from plasma generally pooled from at least 1000 donors.

By “human anti-A polyclonal immunoglobulins” or “anti-A immunoglobulins” is meant human polyclonal immunoglobulins that recognize blood group A antigens. “Blood group A antigens” or “A antigens” are characterized by the presence of a trisaccharide comprising N-acetylgalactosamine (abbreviated hereinafter as “GalNAc”) linked to a galactose (abbreviated hereinafter as “Gal”), which is itself linked to a fucose (abbreviated hereinafter as “Fuc”), according to the following sequence: GalNAcα1-3(Fucα1-2)Gal.

This trisaccharide itself may be attached by its central galactose to other sugars, whose number and assembly vary according to the type of A antigen, as indicated in Table 1 below for A antigen types 1 to 4, and to the presence or absence and the nature of a Lewis antigen.

TABLE 1
Structure of various blood group A antigen types.

| | |
|---|---|
| Type 1 | GalNAcα1-3(Fucα1-2)Galβ1-3GlcNAcβ1-3Galβ1-4Glc-R |
| Type 2 | GalNAcα1-3(Fucα1-2)Galβ1-4GlcNAcβ1-3Galβ1-4Glc-R |
| Type 3 | GalNAcα1-3(Fucα1-2)Galβ1-3GalNAcα1-3Galβ1-4GlcNAc-R |
| Type 4 | GalNAcα1-3(Fucα1-2)Galβ1-3GalNAcβ1-3Galα1-4Gal-R |

GalNAc: N-acetylgalactosamine, Fuc: fucose, Gal: galactose, GlcNAc: N-acetylglucosamine, Glc: glucose, R: support (oligosaccharide, glycoprotein, glycolipid).
The trisaccharide that determines group A is indicated in bold.

By “human anti-B polyclonal immunoglobulins” or “anti-B immunoglobulins” is meant human polyclonal immunoglobulins that recognize blood group B antigens. “Blood group B antigens” or “B antigens” are characterized by the presence of a trisaccharide comprising a first galactose linked to a second galactose, which is itself linked to a fucose, according to the following sequence: Galα1-3(Fucα1-2)Gal.

This trisaccharide itself may be attached by its central galactose to other sugars, whose number and assembly vary according to the type of B antigen, as indicated in Table 2 below for B antigen types 1 to 4, and to the presence or absence and the nature of a Lewis antigen.

TABLE 2
Structure of various blood group B antigen types.

| | |
|---|---|
| Type 1 | Galα1-3(Fucα1-2)Galβ1-3GlcNAcβ1-3Galβ1-4Glc-R |
| Type 2 | Galα1-3(Fucα1-2)Galβ1-4GlcNAcβ1-3Galβ1-4Glc-R |
| Type 3 | Galα1-3(Fucα1-2)Galβ1-3GalNAcα1-3Galβ1-4GlcNAc-R |
| Type 4 | Galα1-3(Fucα1-2)Galβ1-3GalNAcβ1-3Galα1-4Gal-R |

GalNAc: N-acetylgalactosamine, Fuc: fucose, Gal: galactose, GlcNAc: N-acetylglucosamine, Glc: glucose, R: support (oligosaccharide, glycoprotein, glycolipid).
The trisaccharide that determines group B is indicated in bold.

By “human plasma fraction enriched in human polyclonal immunoglobulins” is meant any human plasma fraction able to be obtained by fractionation of human plasma and whose percentage by weight of polyclonal immunoglobulins in relation to the total proteins of the fraction is superior to that of human plasma. Such fractions are advantageously obtained by fractionation of plasma pooled from at least 1000 donors. In particular, they may include any part or subpart of plasma having undergone one or more purification steps, in particular supernatant of cryoprecipitated plasma, plasma cryoprecipitate (resuspended or not), fractions I to V obtained by ethanol fractionation (according to the Cohn method or the Kistler and Nitschmann method), supernatant and precipitate obtained after precipitation with caprylic acid and/or caprylate, filtrates, or any fraction enriched in immunoglobulins (chromatography eluates and/or unadsorbed fractions) by chromatographic separation, as described in particular in WO99/64462 and WO02/092632, and more particularly in WO02/092632.

By “specific ligand of human anti-A polyclonal immunoglobulins” is meant a molecule that binds to human anti-A polyclonal immunoglobulins as defined above and that does not bind to other immunoglobulins. In particular, such ligands may be selected from oligosaccharides representing blood group A antigens. By “oligosaccharide representing blood group A antigens” is meant any oligosaccharide comprising the trisaccharide characteristic of blood group A antigens as defined above: GalNAcα1-3(Fucα1-2)Gal. Such an oligosaccharide may further comprise other sugars present in the blood group A antigens defined above. In particular, in addition to the trisaccharide GalNAcα1-3(Fucα1-2)Gal, such an oligosaccharide may also be selected from tetrasaccharides, pentasaccharides and hexasaccharides derived from the type 1, 2, 3 or 4 group A antigens described above and comprising the characteristic trisaccharide GalNAcα1-3(Fucα1-2)Gal:
Tetrasaccharides:
   Type 1: GalNAcα1-3(Fucα1-2)Galβ1-3GlcNAc,
   Type 2: GalNAcα1-3(Fucα1-2)Galβ1-4GlcNAc,
   Type 3 or 4: GalNAcα1-3(Fucα1-2)Galβ1-3GalNAc,
Pentasaccharides:
   Type 1: GalNAcα1-3(Fucα1-2)Galβ1-3GlcNAc β1-3Gal,
   Type 2: GalNAcα1-3(Fucα1-2)Galβ1-4GlcNAc β1-3Gal,
   Type 3: GalNAcα1-3(Fucα1-2)Galβ1-3GalNAc α1-3Gal,
   Type 4: GalNAcα1-3(Fucα1-2)Galβ1-3GalNAc β1-3Gal,
Hexasaccharides:
   Type 1: GalNAcα1-3(Fucα1-2)Galβ1-3GlcNAc β1-3Galβ1-4Glc,
   Type 2: GalNAcα1-3(Fucα1-2)Galβ1-4GlcNAc β1-3Galβ1-4Glc,
   Type 3: GalNAcα1-3(Fucα1-2)Galβ1-3GalNAc α1-3Galβ1-4GlcNAc,
   Type 4: GalNAcα1-3(Fucα1-2)Galβ1-3GalNAc β1-3Galα1-4Gal.

The oligosaccharide may also comprise repeated units of the characteristic trisaccharide GalNAcα1-3(Fucα1-2)Gal, or tetrasaccharides, pentasaccharides and hexasaccharides derived from the type 1, 2, 3 or 4 group A antigens described above.

Advantageously, the specific ligand of the immunoglobulins that recognize blood group A antigens is the characteristic trisaccharide GalNAcα1-3(Fucα1-2)Gal.

By “specific ligand of human anti-B polyclonal immunoglobulins” is meant a molecule that binds to human anti-B polyclonal immunoglobulins as defined above and that does not bind to other immunoglobulins. In particular, such ligands may be selected from oligosaccharides representing blood group B antigens. By “oligosaccharide representing blood group B antigens” is meant any oligosaccharide comprising the characteristic trisaccharide of blood group B antigens such as defined above: Galα1-3(Fucα1-2)Gal. Such an oligosaccharide may further comprise other sugars present in the blood group B antigens defined above. In particular, in addition to the trisaccharide Galα1-3(Fucα1-2)Gal, such an oligosaccharide may also be selected from tetrasaccharides, pentasaccharides and hexasaccharides derived from the type 1, 2, 3 or 4 group B antigens described above and comprising the characteristic trisaccharide Galα1-3(Fucα1-2)Gal:
Tetrasaccharides:
   Type 1: Galα1-3(Fucα1-2)Galβ1-3GlcNAc,
   Type 2: Galα1-3(Fucα1-2)Galβ1-4GlcNAc,
   Type 3 or 4: Galα1-3(Fucα1-2)Galβ1-3GalNAc,
Pentasaccharides:
   Type 1: Galα1-3(Fucα1-2)Galβ1-3GlcNAc β1-3Gal,
   Type 2: Galα1-3(Fucα1-2)Galβ1-4GlcNAc β1-3Gal,
   Type 3: Galα1-3(Fucα1-2)Galβ1-3GalNAc α1-3Gal,
   Type 4: Galα1-3(Fucα1-2)Galβ1-3GalNAc β1-3Gal,
Hexasaccharides:
Type 1: Galα1-3(Fucα1-2)Galβ1-3GlcNAc β1-3Galβ1-4Glc,
   Type 2: Galα1-3(Fucα1-2)Galβ1-4GlcNAc β1-3Galβ1-4Glc,
   Type 3: Galα1-3(Fucα1-2)Galβ1-3GalNAc α1-3Galβ1-4GlcNAc,
   Type 4: Galα1-3(Fucα1-2)Galβ1-3GalNAc β1-3Galα1-4Gal.

The oligosaccharide may also comprise repeated units of the characteristic trisaccharide Galα1-3(Fucα1-2)Gal, or tetrasaccharides, pentasaccharides and hexasaccharides derived from the type 1, 2, 3 or 4 group B antigens described above.

Advantageously, the specific ligand of the immunoglobulins that recognize blood group B antigens is the characteristic trisaccharide Galα1-3(Fucα1-2)Gal.

Throughout the present description, reference to “a” specific ligand of human anti-A or anti-B polyclonal immunoglobulins includes the possibility of using either a single type of specific ligand of human anti-A or anti-B polyclonal immunoglobulins (I.e., all the ligands grafted onto the support have the same chemical structure), or several distinct types (i.e., different chemical structures) of specific ligands of human anti-A or anti-B polyclonal immunoglobulins. However, it should be understood that each ligand of a given chemical structure that is able to be used is necessarily grafted several times onto the support, in such a way that the human anti-A or anti-B polyclonal immunoglobulins may be retained by the support. The skilled person knows which ligand density must be used in order to allow adsorption of human anti-A or anti-B polyclonal immunoglobulins on the support.

By “autoimmune disease” is meant any disease resulting from an immune system dysfunction which attacks normal body components, called “autoantigens.” Autoimmune diseases, which may be organ-specific or systemic, in particular include peripheral autoimmune thrombocytopenia (in particular idiopathic thrombocytopenic purpura (ITP)), Birdshot retinochoroidopathy, Guillain-Barré syndrome, multifocal motor neuropathy (MMN), chronic demyelinating inflammatory polyradiculoneuropathy (CDIP), Kawasaki's disease, Basedow disease (hyperthyroidism), Hashimoto's thyroiditis (hypothyroidism), systemic lupus erythematosus (SLE), Goodpasture syndrome, pemphigus (in particular pemphigus vulgaris (anti-desmoglein 3)), myasthenia, diabetes due to insulin resistance, autoimmune hemolytic anemia, rheumatoid arthritis, scleroderma, polymyositis, dermatomyositis, Biermer anemia, Gougerot-Sjögren syndrome, glomerulonephritis, Wegener's disease, giant cell arteritis (Horton's disease), periarteritis nodosa, Churg-Strauss syndrome, Still's disease, atrophic polychondritis, Behçet's disease, multiple sclerosis (MS, anti-myelin/axon), spondyloarthritis, Crohn's disease, neuromyelitis optica (anti-AQP4), insulin autoimmune syndrome (IAS), Hirata disease (anti-insulin), Grave's disease, Sydenham's chorea (anti-neuronal protein), Sjögren's syndrome, Muckle-Wells syndrome or Schnitzler syndrome.

By “thrombopenia” or “thrombocytopenia” is meant a decrease in the blood platelet count below the threshold of 150,000 platelets/mm³ or a 50% decrease relative to the subject's reference level. Thrombocytopenia is referred to as “severe” if the blood platelet count drops below the threshold of 100,000 platelets/mm³.

By “peripheral autoimmune thrombocytopenia” is meant thrombocytopenia linked to immunological destruction of platelets. This destruction generally involves synthesis by the subject's B lymphocytes of immunoglobulins against antigens expressed by platelets, whether they are natural platelet antigens or infectious antigens expressed on platelets during infection. The immunoglobulin-coated platelets are then destroyed by effector cells of the immune system, and in particular by macrophages.

Peripheral autoimmune thrombocytopenia includes in particular:
idiopathic thrombocytopenic purpura (ITP).
This autoimmune disease is characterized by the production of antiplatelet immunoglobulins (autoantibodies) in normally healthy subjects not taking any medications. The immunological origin is affirmed by the presence of a large number of immunoglobulins (autoantibodies) on the surface of platelets (see Cines et al. N Engl J Med. 2002 Mar. 28; 346(13):995-1008).
infectious thrombocytopenic purpura.
The mechanism is similar to that of ITP but the causal infection is clearly identified, with immunoglobulins binding to platelets due to their expression of an infectious antigen (see, in particular, Winiarski J et al. Arch Dis Child. 1990 January; 65(1):137-9 with regard to chicken pox; and Kelton et al. J Clin Invest. 1983 April; 71 (4):832-6 with regard to malaria).
immunoallergic drug-induced thrombocytopenic purpura.
The origin of this thrombocytopenic purpura is related to a drug reaction. Many drugs can cause this type of thrombocytopenic purpura (see notably Aster et al. N Engl J Med. 2007 Aug. 9; 357(6):580-7).
neonatal thrombocytopenic purpura.
This thrombocytopenic purpura is related either to transplacental passage of chronic idiopathic thrombocytopenic purpura antibodies, or to fetal/maternal incompatibility leading the production of anti-HPA (Human Platelet Antigen) antibodies (see Peterson et al. Br J Haematol. 2013 April; 161(1):3-14).

By “polycythemia” is meant an abnormal increase in the total volume taken by erythrocytes in the blood. Polycythemia is thus an excess of erythrocytes, which makes the blood more viscous and may cause vascular disorders. Polycythemia can be divided into:
primary polycythemia, due to exacerbated functioning of the bone marrow which produces erythrocytes, generally caused by bone marrow disease such as Vaquez disease also called essential polycythemia, and
polycythemia secondary to hypoxia or to an inappropriate secretion and/or injection of erythropoietin.

By “treatment” is meant improvement, observed on the clinical or biochemical level, in the patient's pathology. In the context of thrombocytopenia, this may notably be observed by an increase in blood platelet count after treatment relative to the platelet count before treatment. In the context of polycythemia, this may notably be observed by a decrease in erythrocyte count after treatment relative to the erythrocyte count before treatment.

By “in combination with another therapeutic agent” is meant the simultaneous or sequential administration of the human anti-A and/or anti-B polyclonal immunoglobulin composition described herein with one or more other therapeutic agent(s), in particular with one or more drugs useful in the treatment of an autoimmune disease and/or a polycythemia. By “simultaneous administration” is meant both the administration in the form of a single pharmaceutical formulation combining the human anti-A and/or anti-B polyclonal immunoglobulin composition described herein and one or more other therapeutic agent(s), as well as the separate administration of at least two distinct pharmaceutical formulations, one containing the human anti-A and/or anti-B polyclonal immunoglobulin composition described herein and the other formulation(s) containing the other therapeutic agent(s), at the same time or at a very short interval (at most 1 hour). By “sequential administration” is meant separate administration of at least two distinct pharmaceutical formulations, one containing the human anti-A and/or anti-B polyclonal immunoglobulin composition described herein and the other formulation(s) containing the other therapeutic agent(s) at an interval greater than 1 hour.

Human Anti-A and/or Anti-B Polyclonal Immunoglobulin Compositions, for Use as a Medicament

### Contents of the Compositions

The present invention relates to a composition comprising human polyclonal immunoglobulins, characterized in that at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of human polyclonal immunoglobulins present in the composition are human anti-A and/or anti-B polyclonal immunoglobulins, for use as a medicament.

The invention also relates to a composition comprising human polyclonal immunoglobulins, characterized in that at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of human polyclonal immunoglobulins present in the composition are human anti-A and/or anti-B polyclonal immunoglobulins, for use as a medicament in the treatment of an autoimmune disease and/or in the treatment of polycythemia.

The invention also relates to the use of a composition comprising human polyclonal immunoglobulins, characterized in that at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of human polyclonal immunoglobulins present in the composition are human anti-A and/or anti-B polyclonal immunoglobulins, for the preparation of a medicament, in particular intended for the treatment of autoimmune diseases (in particular idiopathic thrombocytopenic purpura (ITP)) and/or for the treatment of polycythemia.

The invention also relates to a method for treating autoimmune diseases (in particular idiopathic thrombocytopenic purpura (ITP)) and/or polycythemia in a subject in need thereof, comprising administering to said subject an effective amount of a composition comprising human polyclonal immunoglobulins, characterized in that at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of human polyclonal immunoglobulins present in the composition are human anti-A and/or anti-B polyclonal immunoglobulins.

In an advantageous embodiment, the composition comprises both human anti-A polyclonal immunoglobulins and human anti-B polyclonal immunoglobulins, and the weight ratio of human anti-A polyclonal immunoglobulins to human anti-B polyclonal immunoglobulins (anti-A:anti-B) is comprised between 1:10 and 10:1.

In an embodiment, the composition comprising both human anti-A polyclonal immunoglobulins and human anti-B polyclonal immunoglobulins is enriched in human anti-A polyclonal immunoglobulins, and thus has a weight ratio of human anti-A polyclonal immunoglobulins to human anti-B polyclonal immunoglobulins (anti-A:anti-B) comprised between 2:1 and 10:1.

In another embodiment, the composition comprising both human anti-A polyclonal immunoglobulins and human anti-B polyclonal immunoglobulins is enriched in human anti-B polyclonal immunoglobulins, and thus has a weight ratio of human anti-A polyclonal immunoglobulins to human anti-B polyclonal immunoglobulins (anti-A:anti-B) comprised between 1:10 and 1:2.

In another embodiment, the composition comprising both human anti-A polyclonal immunoglobulins and human anti-B polyclonal immunoglobulins is balanced in terms of the two types of immunoglobulins, and thus has a weight ratio of human anti-A polyclonal immunoglobulins to human anti-B polyclonal immunoglobulins (anti-A:anti-B) comprised between 3:10 and 7:10, advantageously between 4:10 and 6:10.

Alternatively or in combination with the weight percents or ratios mentioned above, the composition enriched in human anti-A polyclonal immunoglobulins according to the invention may have an anti-A activity enriched by a factor of at least 4, advantageously at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 450, at least 500, at least 600, at least 700, at least 800, at least 900, at least 1000, at least 1500, at least 2000, at least 2500, at least 3000, at least 4000, at least 5000, at least 6000, at least 7000, at least 8000, at least 9000, or even at least 10000 as compared to a lyophilized normal human immunoglobulin reference or an EDQM reference (European Directorate for the Quality of Medicines & HealthCare).

Alternatively or in combination with the weight percents or ratios mentioned above, the composition enriched in human anti-B polyclonal immunoglobulins according to the invention may have an anti-B activity enriched by a factor of at least 4, advantageously at least 5, at least 6, at least 7, at least 8, at least 9, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, at least 100, at least 125, at least 150, at least 175, at least 200, at least 250, at least 300, at least 350, at least 400, at least 450, at least 500, at least 600, at least 700, at least 800, at least 900, at least 1000, at least 1500, at least 2000, at least 2500, at least 3000, at least 4000, at least 5000, at least 6000, at least 7000, at least 8000, at least 9000, at least 10000, at least 11000, at least 12000, at least 13000, at least 14000, at least 15000, at least 16000, at least 17000, at least 18000, or even at least 19000 as compared to a lyophilized normal human immunoglobulin reference or to an EDQM reference.

Alternatively or in combination with the weight percents or ratios mentioned above, the composition can comprise both human anti-A polyclonal immunoglobulins and human anti-B polyclonal immunoglobulins and has a ratio (anti-A activity:anti-B activity) comprised between 1:10 and 10:1, in particular between 1:9 and 9:1, between 1:8 and 8:1, between 1:7 and 7:1, between 1:6 and 6:1, between 1:5 and 5:1, between 1:4 and 4:1, between 1:3 and 3:1, or even between 1:2 and 2:1 or between 0.6 and 1.5.

The anti-A activity:anti-B activity ratio and the anti-B activity:anti-A activity ratio are calculated on the basis of anti-A and anti-B activity results obtained in tests carried out in parallel, with the same activity assay method (such as one of those described below, and in particular the flow cytometry method described herein) and expressed in arbitrary units with respect to the same reference standard (EDQM reference standard Y0001688 or a Lyophilized human normal immunoglobulin medicament).

The weight percent of human anti-A and/or anti-B polyclonal immunoglobulins among the total human polyclonal immunoglobulins of a composition comprising purified human polyclonal immunoglobulins may be measured by purifying the composition by affinity chromatography on a column grafted with specific ligands of human anti-A and/or anti-B polyclonal immunoglobulins, and by calculating the ratio between the weight of immunoglobulins adsorbed onto the column and the weight of total immunoglobulins. If the composition is not purified in immunoglobulins, a preliminary step of purification of the total immunoglobulins then makes it possible to measure the weight percent of human anti-A and/or anti-B polyclonal immunoglobulins among the total human polyclonal immunoglobulins.

The compositions for therapeutic use according to the invention are preferably purified, and human polyclonal immunoglobulins advantageously represent at least 85%, advantageously at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of the total proteins of the composition.

The compositions for therapeutic use according to the invention may comprise human polyclonal immunoglobulins of only one isotype (IgG, IgM, IgA, IgD, IgE, advantageously IgG or IgM, preferably IgG) or of several isotypes. However, in a preferred embodiment, the human polyclonal immunoglobulins present in the compositions for therapeutic use according to the invention are mostly (at least 90%, advantageously at least 91%, at least 92%, at least 93%, at least 94%, more advantageously at least 95%, at least 96%, at least 97%, even more advantageously at least 98%, at least 99% by weight) IgG. In this case, the compositions for therapeutic use according to the invention are advantageously enriched in subclass IgG2 immunoglobulins relative to human normal immunoglobulin concentrates. In particular, the IgG compositions for therapeutic use according to the invention are advantageously characterized in that at least 40%, at least 45%, at least 50%, at least 55%, at least 60%, at least 65%, at least 70%, at least 75%, or even at least 80% by weight of human polyclonal immunoglobulins of IgG isotype present in the composition are subclass IgG2 immunoglobulins, advantageously measured by nephelometry and/or spectrography and/or a subclass ELISA kit (i.e., by nephelometry, spectrography, a subclass ELISA kit, or several of these methods, for example by nephelometry and spectrography, or by each of these three methods). Alternatively, or additionally, the IgG compositions according to the invention advantageously have an IgG2:IgG1 weight ratio of at least 0.8, at least 0.9, advantageously at least 1, at least 1.1, at least 1.2, at least 1.3, at least 1.4, at least 1.5, at least 1.6, at least 1.7, at least 1.8, at least 1.9, or even at least 2, at least 2.5, at least 3, at least 3.1, at least 3.2, at least 3.3, at least 3.4, at least 3.5, at least 3.6, at least 3.7, at least 3.8, at least 3.9, or even at least 4, advantageously measured by nephelometry and/or spectrography and/or a subclass ELISA kit (i.e., by nephelometry, spectrography, a subclass ELISA kit, or several of these methods, for example by nephelometry and spectrography, or by each of these three methods).

In another preferred embodiment, the human polyclonal immunoglobulins present in the compositions for therapeutic use according to the invention are mostly (at least 90%, advantageously at least 91%, at least 92%, at least 93%, at least 94%, more advantageously at least 95%, at least 96%, at least 97%, even more advantageously at least 98%, at least 99% by weight) IgM.

The compositions according to the invention are purified and are advantageously concentrated.

Advantageously, the compositions according to the invention are concentrated using any method known to a skilled person, for instance using an ultrafiltration membrane, a centrifugation, a dialysis, or several of these steps.

Advantageously, the concentrated compositions according to the invention have a result in the indirect Coombs test (described below) superior to 1:64, advantageously superior to 1:128, superior to 1:256, superior to 1:512, superior to 1:1024, superior to 1:2048, even superior to 1:4096. By result in the indirect Coombs test superior to 1:N is meant that the result of the indirect Coombs test is negative at a sample dilution of 1:N.

Advantageously, the concentrated compositions according to the invention have a result in the direct agglutination method (described below) superior to 1:64, advantageously superior to 1:128, superior to 1:256, superior to 1:512, superior to 1:1024, superior to 1:2048, or even superior to 1:4096. By result in the direct agglutination test superior to 1:N is meant that the result in the direct agglutination method is negative at a sample dilution of 1:N.

In particular, in an advantageous embodiment, the concentrated compositions according to the invention have a human anti-A and/or anti-B polyclonal immunoglobulin concentration superior to 1 g/L, superior to 1.5 g/L, superior to 2 g/L, superior to 5 g/L, superior to 10 g/L, superior to 15 g/L, superior to 20 g/L, or even superior to 50 g/L.

### Patients Concerned

As explained above, the rationale for the therapeutic efficacy of the compositions for therapeutic use according to the invention for the treatment of autoimmune diseases is based in particular on the fact of saturating macrophages with administered human anti-A and/or anti-B polyclonal immunoglobulin-coated erythrocytes, thus preventing platelet destruction by these same macrophages. Indeed, macrophage Fc receptors are thus saturated with human anti-A and/or anti-B polyclonal immunoglobulin-coated erythrocytes, preventing the binding of platelets coated with other antibodies. Without binding to macrophages, the platelets are not destroyed. For the treatment of polycythemia, the rationale for the therapeutic efficacy is based on the binding of the administered human anti-A and/or anti-B polyclonal immunoglobulins directly to erythrocytes, thus leading to their lysis.

Consequently, it is advantageous to administer the compositions for therapeutic use according to the invention to patients whose erythrocytes carry antigens recognized by the immunoglobulins of the composition. Thus, the compositions for therapeutic use according to the invention are advantageously intended for patients of blood group A (erythrocytes carrying A antigens recognized by human anti-A polyclonal immunoglobulins), B (erythrocytes carrying B antigens recognized by human anti-B polyclonal immunoglobulins) or AB (erythrocytes carrying A and B antigens recognized by human anti-A and anti-B polyclonal immunoglobulins).

Moreover, the rationale for the therapeutic efficacy of the compositions for therapeutic use according to the invention for the treatment of autoimmune diseases is also based on:
a consumption of complement proteins by anti-A and/or anti-B immunoglobulin-coated erythrocytes, reducing the complement-dependent activity of pathogenic autoantibodies, making it possible to treat autoimmune pathologies for which the role of the complement is harmful to the patient, such as, for example, neuromyelitis optica (anti-AQP4) and multiple sclerosis (anti-myelin/axon).
an anti-inflammatory action of anti-A and/or anti-B immunoglobulin-coated erythrocytes due to the inhibition induced during their phagocytosis of proinflammatory cytokine secretion by activated macrophages, making it possible to treat autoimmune pathologies for which effector cells expressing Fc receptors, in particular CD16, are activated and maintain a pathogenic inflammatory state in the patient.
an immunomodulatory action of anti-A and/or anti-B immunoglobulin-coated erythrocytes which, by limiting cell destruction allow for the induction of a negative signal and thus a decrease in autoantibody secretion by cross-linking between BCR and FcgRIIb of the B cell via the autoantibody and the antigen expressed on the target cell surface, making it possible to treat autoimmune pathologies for which a pathogenic autoantibody is shown.
an induction of anti-inflammatory cytokines such as IL1-RA by immune system cells, epithelial cells and adipocytes induced by anti-A and/or anti-B immunoglobulin-coated erythrocytes, making it possible to treat inflammatory pathologies.

In particular, when the compositions for therapeutic use according to the invention comprise both human anti-A and human anti-B polyclonal immunoglobulins, they are advantageously intended for blood group A, B or AB patients, and in particular blood group AB patients.

### Autoimmune Diseases Concerned

The compositions for therapeutic use according to the invention may be used in the treatment of autoimmune diseases.

The compositions for therapeutic use according to the invention may notably be used in the treatment of peripheral autoimmune thrombocytopenia (involving an immunological destruction of platelets, in particular ITP), Birdshot retinochoroidopathy, Guillain-Barré syndrome, multifocal motor neuropathy (MMN), chronic demyelinating inflammatory polyradiculoneuropathies (CDIP), Kawasaki's disease, Basedow disease (hyperthyroidism), Hashimoto's thyroiditis (hypothyroidism), systemic lupus erythematosus (SLE), Goodpasture syndrome, pemphigus (in particular pemphigus vulgaris (anti-desmoglein 3)), myasthenia, diabetes due to insulin resistance, autoimmune hemolytic anemia, rheumatoid arthritis, scleroderma, polymyositis, dermatomyositis, Biermer anemia, Gougerot-Sjögren syndrome, glomerulonephritis, Wegener's disease, giant cell arteritis (Horton's disease), periarteritis nodosa, Churg-Strauss syndrome, Still's disease, atrophic polychondritis, Behçet's disease, multiple sclerosis (MS, anti-myelin/axon), spondyloarthritis, Crohn's disease, neuromyelitis optica (anti-AQP4), insulin autoimmune syndrome (IAS), Hirata disease (anti-insulin), Grave's disease, Sydenham's chorea (anti-neuronal protein), Sjögren's syndrome, Muckle-Wells syndrome or Schnitzler syndrome. Indeed, several mechanisms make it possible to use the composition according to the invention to treat autoimmune diseases.

Advantageously, the autoimmune diseases for which the role of the complement is harmful to the patient, such as, for example, neuromyelitis optica (anti-AQP4) and multiple sclerosis (anti-myelin/axon), may be treated by the composition according to the invention thanks to the mechanism of consumption of complement proteins by anti-A and/or anti-B immunoglobulin-coated erythrocytes.

Advantageously, the autoimmune diseases for which effector cells expressing Fc receptors, in particular CD16, are activated and maintain a pathogenic inflammatory state in the patient may be treated by the composition according to the invention thanks to the anti-inflammatory action of anti-A and/or anti-B immunoglobulin-coated erythrocytes.

Advantageously, the autoimmune diseases for which a pathogenic autoantibody is shown, such as ITP, insulin autoimmune syndrome (IAS), Hirata disease (anti-insulin), neuromyelitis optica (anti-AQP4), Grave's disease, pemphigus, multiple sclerosis (anti-myelin/axon), Sydenham's chorea (anti-neuronal protein), Sjögren's syndrome or pemphigus vulgaris (anti-desmoglein 3), may be treated with the composition according to the invention thanks to the immunomodulatory action of anti-A and/or anti-B immunoglobulin-coated erythrocytes.

Advantageously, inflammatory autoimmune diseases such as rheumatoid arthritis or for example Muckle-Wells syndrome and Schnitzter syndrome may be treated with the composition according to the invention thanks to the mechanism of induction of anti-inflammatory cytokines such as IL1-RA by immune system cells.

In particular, the compositions for therapeutic use according to the invention may be used in the treatment of peripheral autoimmune thrombocytopenia, involving an immunological destruction of platelets.

Indeed, as explained above, the rationale for the therapeutic efficacy of the compositions for therapeutic use according to the invention is notably based on saturating macrophages with administered human anti-A and/or anti-B polyclonal immunoglobulin-coated erythrocytes, thus preventing platelet destruction by these same macrophages. Moreover, the mechanisms of consumption of complement proteins by anti-A and/or anti-B immunoglobulin-coated erythrocytes, of anti-inflammatory action of anti-A and/or anti-B immunoglobulin-coated erythrocytes, of immunomodulatory action of anti-A and/or anti-B immunoglobulin-coated erythrocytes, and of induction of anti-inflammatory cytokines such as IL1-RA by immune system cells, also make it possible to prevent platelet destruction.

The compositions for therapeutic use according to the invention are notably advantageously used in the treatment of idiopathic thrombocytopenic purpura (ITP), infectious thrombocytopenic purpura, immunoallergic drug-induced thrombocytopenic purpura, or neonatal thrombocytopenic purpura, and in particular in the treatment of idiopathic thrombocytopenic purpura (ITP).

### Polycythemia-Type Diseases Concerned

The compositions for therapeutic use according to the invention may be used in the treatment of polycythemia.

In particular, the compositions for therapeutic use according to the invention may be used in the treatment of polycythemia, involving an increase in erythrocyte count.

Indeed, as explained above, the rationale for therapeutic efficacy is based on the binding of administered human anti-A and/or anti-B polyclonal immunoglobulins directly to erythrocytes, thus leading to their lysis.

The compositions for therapeutic use according to the invention are notably advantageously used in the treatment of primary polycythemia, in particular in Vaquez disease (also called essential polycythemia), and/or in the treatment of secondary polycythemia due to hypoxia or due to the inappropriate secretion and/or injection of erythropoietin.

### Therapeutic Dose and Administration

It is important to use an appropriate dose of the compositions for therapeutic use according to the invention.

Indeed, administration of the compositions according to the invention necessarily involves the destruction of a certain number of erythrocytes (hemolysis), and thus it is advisable not to over-dose the compositions for therapeutic use according to the invention, in order to avoid excessive hemolysis, which is also harmful to the treated subject. That is also why anti-A and anti-B immunoglobulins levels present in therapeutic human polyclonal immunoglobulin concentrates are limited to maximum values by health authorities. In particular, when therapeutic human polyclonal immunoglobulin concentrates are used in replacement therapy in primary or secondary immunodeficiencies with an antibody production defect, it is advisable to minimize any risk of hemolysis.

However, in the case of autoimmune diseases and in particular peripheral autoimmune thrombocytopenia and/or polycythemia, moderate controlled hemolysis may be acceptable and beneficial to the patient.

To obtain moderate hemolysis, the dose of anti-A and/or anti-B immunoglobulins administered to a patient with polycythemia and/or an autoimmune disease, and in particular with peripheral autoimmune thrombocytopenia (in particular ITP), should preferably be comprised between 20 and 100 μg/kg body weight, advantageously between 25 and 90 μg/kg body weight, between 30 and 80 μg/kg body weight, between 35 and 70 μg/kg body weight, between 40 and 60 μg/kg body weight, between 45 and 55 μg/kg body weight, and in particular about 50 μg/kg body weight.

The doses are advantageously adjusted so as to avoid severe hemolysis in the patient, which may cause serious and/or harmful side effects.

Since the composition for therapeutic use according to the invention is intended to bind to erythrocytes, it is advantageously administered via the intravenous route. In another embodiment, the composition for therapeutic use according to the invention is administered via the enteral, parenteral, local and mucocutaneous routes.

The administration rate may be about 1 to 5 mL (in particular 1 to 4 mL, 1 to 3 mL, or about 2 mL) of a composition comprising 150 mg/L anti-A and/or anti-B immunoglobulins every 15 to 60 seconds.

The composition for therapeutic use according to the invention may also be administered in combination with another therapeutic agent selected from the drugs useful in the treatment of an autoimmune disease and/or a polycythemia.

In an advantageous embodiment, when the autoimmune disease is peripheral autoimmune thrombocytopenia (and in particular ITP), the frequency of administration of the composition for therapeutic use according to the invention is adjusted according to the platelet count to be reached and/or maintained in the patient.

### Preparation of the Compositions for Therapeutic Use According to the Invention

The compositions for therapeutic use according to the invention may be obtained from varyingly purified fractions of human plasma comprising polyclonal immunoglobulins using various purification methods, and in particular by a method comprising the following steps:
a) adsorbing a batch of human plasma or a human plasma fraction enriched in human polyclonal immunoglobulins on a support grafted with a specific ligand of human anti-A polyclonal immunoglobulins and/or with a specific ligand of human anti-B polyclonal immunoglobulins,
b) storing the unadsorbed fraction for possible later use, and
c) dissociating and collecting the adsorbed fraction.

Preparation of the compositions for therapeutic use according to the invention is based on a step of specific adsorption of human anti-A and/or anti-B polyclonal immunoglobulins on a support grafted with a specific ligand of said immunoglobulins, which are then eluted. Advantageously, it is possible to start directly from plasma or from a human plasma fraction enriched in human polyclonal immunoglobulins, varyingly purified for human polyclonal immunoglobulins.

In a particular embodiment, the method for preparing the compositions for therapeutic use according to the invention may be integrated into a more general method for purifying therapeutic human normal immunoglobulins (collection of heretofore discarded fractions) and may thus be used on a pre-purified human polyclonal immunoglobulin fraction. Said pre-purified human polyclonal immunoglobulin fraction advantageously has a human polyclonal immunoglobulin content of at least 80%, advantageously at least 81%, at least 82%, more advantageously at least 83%, at least 84%, even more advantageously at least 85%, at least 86%, at least 87%, at least 88%, at least 89%, or even at least 90%, at least 91%, or at least 92% by weight of the total proteins of the fraction.

As indicated above, the pre-purified human polyclonal immunoglobulin fraction may in particular have been obtained by chromatographic separation, in particular according to the human polyclonal immunoglobulin purification methods described in WO99/64462 and WO02/092632, and more particularly in WO02/092632. In this case, the pre-purified human polyclonal immunoglobulin fraction is obtained via pre-purification by a step of precipitation of lipid contaminants of blood plasma or of an IgG-enriched blood plasma fraction, and a single step of chromatography on an anion-exchange resin carried out at alkaline pH, with selective elution of IgG in one step using a suitable buffer at a pH comprised between 4 to 7. Advantageously, pre-purification using a step of precipitation of lipid contaminants consists of a caprylic acid precipitation step.

When a pre-purified human polyclonal immunoglobulin fraction is used in step a) of the method described above, the pre-purified human polyclonal immunoglobulin fraction may also have undergone a biological safety step (virus removal and/or virus inactivation, in particular by solvent-detergent treatment), a concentration step (in particular by ultrafiltration), and/or a sterilizing filtration step.

In a method for preparing a composition for therapeutic use according to the invention, the support may be any suitable support likely to be selected by the skilled person for adsorbing human anti-A and/or anti-B polyclonal immunoglobulins.

Such a support used in step a) is advantageously in the form of:
a) particles (in particular polymer particles) grafted with the ligand(s) of interest, or
b) a polymer membrane, the membrane being grafted with the ligand(s) of interest.

The support may thus in particular be in the form of particles grafted with the ligand(s) of interest. The particles are advantageously spherical or oblong in shape, and in particular may be beads. Said particles generally have a mean size of about 0.1 μm to about 1000 μm, preferably of about 20 μm to about 500 μm, more preferably of about 50 μm to about 200 μm, still more preferably of about 70 μm to about 120 μm. They may consist of polymer or of inorganic matter (such as silica or glass, for example). Advantageously, the particles are porous.

In an advantageous embodiment, they are polymer particles. The polymer may be natural or non-natural (synthetic or semisynthetic), organic or inorganic (preferably the polymer will be organic), cross-linked or not cross-linked (preferably the polymer will be cross-linked). Advantageously, the polymer is a cross-linked organic polymer.

The polymer may in particular be selected from cellulose and derivatives thereof, agarose, dextran, polyacrylates, polystyrene, polyacrylamide, polymethacrylamide, styrene and divinylbenzene copolymers, or mixtures of said polymers.

In a preferred embodiment, the polymer is cellulose, and the particles are preferably porous cellulose beads. More preferably still, it is cross-linked cellulose.

The support may also be in the form a polymer membrane, the membrane being grafted with the ligand(s) of interest. The membrane polymer may be selected from the polymers mentioned above for polymer particles.

The particles are advantageously incorporated into a gel or a resin, which is used as the matrix in an affinity chromatography column. In the same way, the polymer membrane may be included in an affinity chromatography column. The batch of human plasma or the human plasma fraction enriched in human polyclonal immunoglobulins is then adsorbed on the affinity chromatography column, and the adsorbed fraction is eluted and collected. However, although preferred, the use of an affinity chromatography column is not essential, and other methods of adsorption, dissociation and collection may be used.

In a method for preparing a composition for therapeutic use according to the invention, the specific ligand of human anti-A polyclonal immunoglobulins may be any suitable molecule known to the skilled person that binds to human anti-A polyclonal immunoglobulins as defined above and that does not bind to other immunoglobulins. Such a ligand is advantageously selected from oligosaccharides representative of type 1, 2, 3 and 4 group A antigens and in particular from the following oligosaccharides:
Trisaccharide: GalNAcα1-3(Fucα1-2)Gal;
Tetrasaccharides:
   Type 1: GalNAcα1-3(Fucα1-2)Galβ1-3GlcNAc,
   Type 2: GalNAcα1-3(Fucα1-2)Galβ1-4GlcNAc,
   Type 3 or 4: GalNAcα1-3(Fucα1-2)Galβ1-3GalNAc
Pentasaccharides:
   Type 1: GalNAcα1-3(Fucα1-2)Galβ1-3GlcNAc β1-3Gal,
   Type 2: GalNAcα1-3(Fucα1-2)Galβ1-4GlcNAc β1-3Gal,
   Type 3: GalNAcα1-3(Fucα1-2)Galβ1-3GalNAc α1-3Gal,
   Type 4: GalNAcα1-3(Fucα1-2)Galβ1-3GalNAc β1-3Gal,
Hexasaccharides:
   Type 1: GalNAcα1-3(Fucα1-2)Galβ1-3GlcNAc β1-3Galβ1-4Glc,
   Type 2: GalNAcα1-3(Fucα1-2)Galβ1-4GlcNAc β1-3Galβ1-4Glc,
   Type 3: GalNAcα1-3(Fucα1-2)Galβ1-3GalNAc α1-3Galβ1-4GlcNAc,
   Type 4: GalNAcα1-3(Fucα1-2)Galβ1-3GalNAc β1-3Galα1-4Gal.

In a method for preparing a composition for therapeutic use according to the invention, the specific ligand of human anti-B polyclonal immunoglobulins may be any suitable molecule known to the skilled person that binds to human anti-B polyclonal immunoglobulins as defined above and that does not bind to other immunoglobulins.

Such a ligand is advantageously selected from oligosaccharides representative of type 1, 2, 3 and 4 group B antigens and in particular from the following oligosaccharides:
Trisaccharide: Galα1-3(Fucα1-2)Gal;
Tetrasaccharides:
   Type 1: Galα1-3(Fucα1-2)Galβ1-3GlcNAc,
   Type 2: Galα1-3(Fucα1-2)Galβ1-4GlcNAc,
   Type 3 or 4: Galα1-3(Fucα1-2)Galβ1-3GalNAc,
Pentasaccharides:
   Type 1: Galα1-3(Fucα1-2)Galβ1-3GlcNAc β1-3Gal,
   Type 2: Galα1-3(Fucα1-2)Galβ1-4GlcNAc β1-3Gal,
   Type 3: Galα1-3(Fucα1-2)Galβ1-3GalNAc α1-3Gal,
   Type 4: Galα1-3(Fucα1-2)Galβ1-3GalNAc β1-3Gal,
Hexasaccharides:
   Type 1: Galα1-3(Fucα1-2)Galβ1-3GlcNAc β1-3Galβ1-4Glc,
   Type 2: Galα1-3(Fucα1-2)Galβ1-4GlcNAc β1-3Galβ1-4Glc,
   Type 3: Galα1-3(Fucα1-2)Galβ1-3GalNAc α1-3Galβ1-4GlcNAc,
   Type 4: Galα1-3(Fucα1-2)Galβ1-3GalNAc β1-3Galα1-4Gal.

In step a) of the method as described above, the support may be grafted with a specific ligand of human anti-A polyclonal immunoglobulins and/or with a specific ligand of human anti-B polyclonal immunoglobulins.

In an embodiment, the support is grafted only with a specific ligand of human anti-A polyclonal immunoglobulins.

In another embodiment, the support is grafted only with a specific ligand of human anti-B polyclonal immunoglobulins.

In yet another embodiment, the support is grafted both with a specific ligand of human anti-A polyclonal immunoglobulins and with a specific ligand of human anti-B polyclonal immunoglobulins. In this case, a mixture of supports grafted with a specific ligand of human anti-A polyclonal immunoglobulins and of supports grafted with a specific ligand of human anti-B polyclonal immunoglobulins, in respective proportions generally comprised between 25:75 (v/v) and 75:25 (v/v), and in particular of 50:50 (v/v), will advantageously be used. In particular, when particles grafted with the ligand(s) of interest are used, a mixture of particles grafted with a specific ligand of human anti-A polyclonal immunoglobulins and of particles grafted with a specific ligand of human anti-B polyclonal immunoglobulins may be used to prepare a gel and to fill an affinity chromatography column. In this case, particles grafted with a specific ligand of human anti-A polyclonal immunoglobulins and particles grafted with a specific ligand of human anti-B polyclonal immunoglobulins are mixed in respective proportions generally comprised between 25:75 (v/v) and 75:25 (v/v), and in particular of 50:50 (v/v). In another embodiment, it is also possible to use a support comprising particles grafted with both a specific ligand of human anti-A polyclonal immunoglobulins and a specific ligand of human anti-B polyclonal immunoglobulins. In another embodiment, it is also possible to use a mixture of:
particles grafted with both a specific ligand of human anti-A polyclonal immunoglobulins and a specific ligand of human anti-B polyclonal immunoglobulins, and
particles grafted with a specific ligand of human anti-A polyclonal immunoglobulins and/or particles grafted with a specific ligand of human anti-B polyclonal immunoglobulins.

In a method for preparing a composition for therapeutic use according to the invention, the ligand of interest is advantageously grafted onto the polymer particles or onto the polymer membrane via a spacer, which reduces steric hindrance and makes the trisaccharide characteristic of A or B antigens more accessible to immunoglobulins able to be adsorbed on the support.

Such a spacer may be any suitable group known to the skilled person that allows the ligand of interest, and thus oligosaccharides in particular, to be grafted onto a support of interest, in particular the polymers described above.

The spacer typically comprises at least one C, O, N, or S atom, and will generally comprise at least one of the following chemical functional groups: ether (—O—), thioether (—S—), amino (—NH—), carboxy —(—COO— or —OCO—), amide (—CONH— or —HNOC—).

It may in particular be selected from:
—(CH₂)mX(CH₂)n- or —(CH₂)mX1(CH₂)nX2(CH₂)p-, wherein X, X1, and X2 are each independently selected from O, S, NH and a covalent bond; m, n, and p are each independently 0, 1, 2, 3, 4, 5 or 6; and 1, 2 or 3 of the hydrogen atoms may be replaced by an equivalent number of OH and/or methyl groups.
The spacer may in particular have a structure selected from:

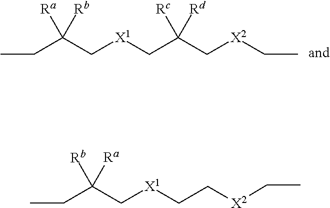

wherein each of X1 and X2 is independently selected from O, S, and NH; and each of Ra, Rb, Rc, and Rd is independently selected from H, OH, and methyl.
One of the following structures:

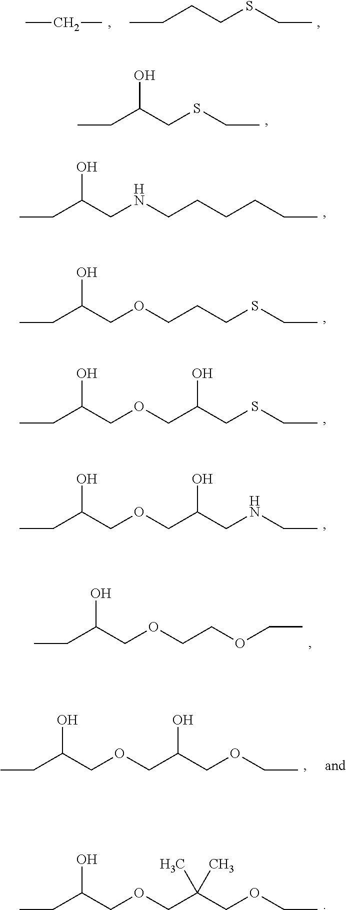

Spacers of formula —NH—R1-CONH—R2-, wherein R1 is a C₄-C₆ alkyl group, R2 is a C₃-C₈ alkyl group, and said spacer is linked by its amine functional group (in bold above) to the support.
In this case, R1 is a linear or branched, preferably linear C₄-C₆ alkyl group. Preferably, R1 is a C₅ alkyl group.
R2 is a linear or branched, preferably linear C₃-C₈ alkyl group. Preferably, R2 is a C₃ alkyl group.
In a preferred embodiment, the ligands (which are preferably trisaccharides as described above) are grafted onto the particles or the membrane via a spacer of formula: (particle/membrane)-NH—C₅H₁₀—CO—NH—C₃H₆— (ligand).

Coupling between the particle or the membrane and the spacer, on the one hand, and coupling between the spacer and the specific ligand of human anti-A polyclonal immunoglobulins or the specific ligand of human anti-B polyclonal immunoglobulins, on the other, may be carried out by any suitable chemical synthesis protocol known to the skilled person.

In a particular embodiment, the particle or the membrane may carry an —NH—R1-COOH arm. Preferably, said arm is ε-aminocaproic acid (wherein R1 is a pentyl group). Conventionally, the particle may then be activated using bifunctional reagents such as epichlorohydrin, epibromohydrin, dibromo- and dichloropropanol, dibromobutane, ethylene glycol diglycidyl ether, butanediol diglycidyl ether, divinyl sulfone, allyl glycidyl ether, and allyl bromide. The bifunctional reagent is able to react with both the particles/membrane and the —NH—R1-COOH arm. Heterofunctional allylic compounds, such as allyl bromide, are preferred bifunctional reagents and make it possible to produce an activated matrix.

For certain solid supports, such as cellulose, composites containing hydrogel, or other materials having hydroxyl groups, it is advantageous to deprotonate the hydroxyl groups with a hydroxide source, for example, before reaction with a bifunctional reagent.

The ligands representing antigens of blood groups A and/or B are then immobilized on the activated particle/membrane carrying the —NH—R1-COOH arm via an —NH—R2- linking group, wherein R2 is a linear or branched, preferably linear C₃-C₈ alkyl group. To that end, the COOH functional group of the —NH—R1-COOH arm carried by the particle/membrane is reacted with the NH₂ functional group of the NH₂—R2-oligosaccharide ligand, by use of an N-ethoxycarbonyl-2-ethoxy-1,2-dihydroquinoline (EEDQ)-type condensation agent.

Examples of supports grafted with a specific ligand of human anti-A polyclonal immunoglobulins that may be used in the context of the invention are as follows: Glycosorb ABO A (Sepharose matrix to which the trisaccharide characteristic of A antigen is grafted, Glycorex Transplantation AB, Lund, Sweden), Allotran A (trisaccharide characteristic of A antigen grafted onto a Sepharose FF matrix via polyacrylamide, Lectinity Corp), HyperCel IsoA (cross-linked cellulose particles grafted with the trisaccharide characteristic of A antigen, Pall).

Examples of supports grafted with a specific ligand of human anti-B polyclonal immunoglobulins that may be used in the context of the invention are as follows: Glycosorb ABO B (Sepharose matrix to which the trisaccharide characteristic of B antigen is grafted, Glycorex Transplantation AB, Lund, Sweden), Allotran B (trisaccharide characteristic of B antigen grafted onto a Sepharose FF matrix via polyacrylamide, Lectinity Corp), HyperCel IsoB (cross-linked cellulose particles grafted with the trisaccharide characteristic of B antigen, Pall).

When one wishes to use a support grafted with a specific ligand of human anti-A polyclonal immunoglobulins and a specific ligand of human anti-B polyclonal immunoglobulins, a mixture of a support grafted with a specific ligand of human anti-A polyclonal immunoglobulins as described above and a support grafted with a specific ligand of human anti-B polyclonal immunoglobulins as described above will generally be used. In particular, when particles grafted with the ligand(s) of interest are used, a mixture of particles grafted with a specific ligand of human anti-A polyclonal immunoglobulins and of particles grafted with a specific ligand of human anti-B polyclonal immunoglobulins may be used to prepare a gel and to fill an affinity chromatography column. Particles grafted with both a specific ligand of human anti-A polyclonal immunoglobulins and a specific ligand of human anti-B polyclonal immunoglobulins may also be used to prepare a gel and to fill an affinity chromatography column. It is also possible to use a mixture of:
particles grafted with both a specific ligand of human anti-A polyclonal immunoglobulins and a specific ligand of human anti-B polyclonal immunoglobulins, and
particles grafted with a specific ligand of human anti-A polyclonal immunoglobulins and/or particles grafted with a specific ligand of human anti-B polyclonal immunoglobulins.

When purification of the composition for therapeutic use according to the invention is carried out by affinity chromatography, the batch of human plasma or the human plasma fraction enriched in human polyclonal immunoglobulins is adsorbed in step a) on the chromatography column under any suitable condition known to the skilled person, in particular any condition recommended by the manufacturer of the chromatography support, depending on the support selected. In particular, the batch of human plasma or the human plasma fraction enriched in human polyclonal immunoglobulins may be percolated on the column. The unadsorbed fraction is advantageously collected for other later uses. The adsorbed fraction is then dissociated and collected using one or more washes of the column with one or more suitable elution buffers. In particular, acidic elution buffer (glycine-HCl buffer, pH 2 to 4, for example) and/or basic elution buffer (glycine-NaOH solution, pH 10 to 12, for example) may be used.

Furthermore, the composition thus obtained may undergo one or more subsequent optional steps, such as: a step of neutralization of the composition (adjustment of pH between 3 and 9, preferably between 4 and 5), one or more additional purification steps, a concentration step (by ultrafiltration, for example), at least one inactivation step (solvent-detergent treatment, for example) or virus removal step (nanofiltration, for example), or a combination of several of these steps.

The method as described above enables the obtention of a composition of human polyclonal immunoglobulins of which at least 80%, at least 81%, at least 82%, at least 83%, at least 84%, advantageously at least 85%, at least 86%, at least 87%, more advantageously at least 88%, at least 89%, even more advantageously at least 90%, at least 91%, at least 92%, at least 93%, at least 94%, or even at least 95%, at least 96%, at least 97%, at least 98%, or at least 99% by weight of the polyclonal human immunoglobulins present in the composition recognize antigens of blood groups A and B.

When the support used in step a) is grafted with both a specific ligand of human anti-A polyclonal immunoglobulins and a specific ligand of human anti-B polyclonal immunoglobulins, the purified composition then comprises a mixture of human anti-A polyclonal immunoglobulins and of human anti-B polyclonal immunoglobulins. In human polyclonal immunoglobulins purified from plasma pools, the proportion of human anti-A polyclonal immunoglobulins and human anti-B polyclonal immunoglobulins depends on the initial donor population. Indeed, differences in distribution percentage of the various blood groups exist according to the donor populations. These differences may thus be found in the compositions of human anti-A polyclonal immunoglobulins and/or of human anti-B polyclonal immunoglobulins according to the invention.

When the support used in step a) is grafted with only a specific ligand of human anti-A polyclonal immunoglobulins, the human anti-A polyclonal immunoglobulins are retained, and the composition obtained then comprises human anti-A polyclonal immunoglobulins. Alternatively, when the support used in step a) is grafted with only a specific ligand of human anti-B polyclonal immunoglobulins, the human anti-B polyclonal immunoglobulins are retained, and the composition obtained then comprises human anti-B polyclonal immunoglobulins.

The invention further relates to a composition able to be obtained by one of the preparation methods described above, for use as a medicament, in particular in the treatment of autoimmune diseases (in particular peripheral autoimmune thrombocytopenia) and/or polycythemia (primary or secondary).

The following examples aim to illustrate the present invention.

### EXAMPLES

### Example 1: Preparation of a Human Polyclonal Immunoglobulin Composition, Enriched in Human Anti-A and Anti-B Polyclonal Immunoglobulins

A first human polyclonal immunoglobulin composition according to the invention, enriched in human anti-A and anti-B polyclonal immunoglobulins, was prepared.

### Materials and Methods

A purified human polyclonal immunoglobulin composition was prepared from a plasma pool according to the method described in application WO02/092632.

This purified human polyclonal immunoglobulin composition was then adsorbed on a 1 ml affinity chromatography column filled with a gel comprising a mixture of porous cross-linked cellulose beads grafted with the trisaccharide characteristic of group A antigens (column A) and of porous cross-linked cellulose beads grafted with the trisaccharide characteristic of group B antigens (column B), in respective proportions of 50:50. The load was 1.8 kg of purified human polyclonal immunoglobulin composition per liter of gel. Contact time was set to 2 minutes.
The unadsorbed fraction is collected for subsequent processing in order to prepare a therapeutic human polyclonal immunoglobulin concentrate lacking human anti-A and anti-B polyclonal immunoglobulins.

The fraction of interest in the context of the present invention is then obtained by combining two elution fractions:
A first elution of the chromatography column with acidic buffer (0.1 M glycine, pH 3),
A second elution of the chromatography column with basic buffer (0.1 M glycine, pH 11),
   followed by neutralization of the combined fraction (pH adjusted to 7).

This composition was then analyzed using standard technologies in order to determine the concentrations of IgG, IgA and IgM and the levels of polymers, dimers, monomers and immunoglobulin fragments.

The anti-A and anti-B activity of the composition was also analyzed by the method described in WO2007/077365 and compared with that of a lyophilized human normal immunoglobulin.

### Results

The starting purified human polyclonal immunoglobulin composition, which was adsorbed on the anti-A and anti-B affinity chromatography column, has the following characteristics:
Total proteins: 10.0 g/l
IgG: 9.20 g/l (Subclasses: IgG1: 65%; IgG2: 30%; IgG3: 3%; IgG4: 2%)
IgA: <0.013 g/l
IgM: <0.009 g/l
Molecular size distribution (MSD):
   Polymers: <0.4%
   Dimers: 5.4%
   Monomers: 93.5%
   Fragments: <1.0%

Following the anti-A and anti-B affinity chromatography step, the combined fraction of the two successive elutions with acidic buffer and then with basic buffer has the following characteristics:
IgG: 0.34 g/l
IgA: 0.015 g/l
IgM: <6.3 mg/I
Anti-A: 622.3 AU*
Anti-B: 638.7 AU*
Molecular size distribution (MSD):
   Polymers: 0.1%
   Dimers: 2.9%
   Monomers: 95.6%
   Fragments: 1.3%

At this stage, anti-A activity and anti-B activity are expressed in arbitrary units (AU) with respect to a lyophilized human normal immunoglobulin, the product considered to be the reference whose value is set to 1. Lyophilized human normal immunoglobulin thus has for anti-A and anti-B a negative result in the direct Coombs test at a 1:64 dilution as required by regulatory authorities. Thus, the composition obtained by the method according to the invention has anti-A activity and anti-B activity that is about 600 times superior to that of the human normal polyvalent immunoglobulins of the lyophilized human normal immunoglobulin (therapeutic human polyclonal immunoglobulin concentrate).

### Conclusions

Results presented above show that it is possible to obtain a purified human anti-A and anti-B polyclonal immunoglobulin composition by collecting the fraction of a human polyclonal immunoglobulin composition on an affinity chromatography column carrying Ligands that specifically recognize anti-A and anti-B antibodies.

### Example 2: Therapeutic Efficacy of Anti-A and/or Anti-B Polyclonal Immunoglobulins in the Treatment of Idiopathic Thrombocytopenic Purpura (ITP) in a Murine Model

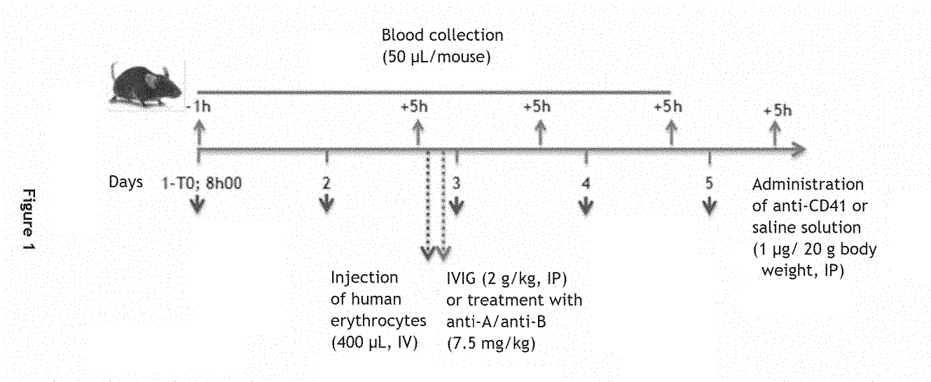

The therapeutic efficacy of anti-A and/or anti-B polyclonal immunoglobulins in the treatment of idiopathic thrombocytopenic purpura (ITP) was tested in a murine model. In this model, mice are injected with platelet-targeting anti-CD41 antibodies in order to induce ITP. In order to test the therapeutic efficacy of anti-A and/or anti-B polyclonal immunoglobulins in group A, B or AB patients, the mice are further injected with human group AB erythrocytes and with anti-A and anti-B polyclonal immunoglobulins (see FIG. 1).

### Materials and Methods

A general diagram of the study is presented in FIG. 1.

### Mice

Eight-week-old female C57BL/6j mice (Janvier, France) were used.

### Induction of ITP

Test C57BL/6j mice (groups 2-5) were injected with 1 μg/20 g body weight of anti-CD41 antibody which deplete platelets (clone MW Reg 30, #3214555, BD Biosciences, San Jose, Calif., USA), diluted in 100 μL/20 g body weight of PBS (#14190-094, Invitrogen, France) (quantity and injection volume were adapted to the mice's weight), via the intraperitoneal (IP) route from days 1 to 5.

Control mice (group 1) were injected with saline solution (0.09% NaCl).

### Preparation and Administration of IVIG

IVIG (LFB) were diluted in 1×PBS to a working concentration of 100 mg/mL (1 g/kg). C57BL/6j mice (group 5) were injected with 2 g/kg of IVIG (with erythrocytes), via the intraperitoneal route on day 2 after anti-CD41 injection. Injection volume was adapted to the mice's weight.

### Administration of Human Erythrocytes

Human group AB+ erythrocytes in isotonic glucose NaCl solution were diluted to 1:4 in 0.09% NaCl (300 μL+100 μL) and administered to groups 3-5 via the intravenous route on day 2, 3 hours after blood collection for hematology analysis (corresponding to 8 hours after anti-CD41 injection on day 2).

### Administration of Anti-A/Anti-B Antibodies

Human anti-A/anti-B antibodies were prepared at the required dose (injection of 100 μL of anti-A anti-B at 7.5 mg/kg in 0.1 M glycine at pH 6) and administered to group 4 via the intraperitoneal route on day 2, 30 minutes after injection of human erythrocytes (corresponding to 8.5 hours after anti-CD41 injection on day 2).

### Experimental Groups

The experimental groups are summarized in Table 3 below:

TABLE 3
Summary of the experimental groups.

| | Anti-CD41 | | |
|---|---|---|---|
| Group no. | Induction (IV) | Treatment | |
| 1 | — | 200 μL vehicle | |
| | | (0.09% NaCl) | |
| 2 | (1 μg/20 g) | 200 μL vehicle | |
| | | (0.09% NaCl) | |
| 3 | (1 μg/20 g) | Human AB + | 200 μL glycine buffer |
| | | erythrocytes (IV) | (0.1M, pH 6) (IP) |
| 4 | (1 μg/20 g) | Human AB + | +100 μL anti-A/anti-B |
| | | erythrocytes (IV) | (7.5 mg/kg) (IP) |
| 5 | (1 μg/20 g) | Human AB + | +200 μL IVIG |
| | | erythrocytes (IV) | (2 g/kg) (IP) |

IP: intraperitoneal, IV: intravenous.

### Blood Collection

Blood (50 μL) was collected daily by retro-orbital puncture under 3% isoflurane anesthesia (DDG9623, #11K10A33, Baxter, France) in tubes (containing ethylenediaminetetraacetic acid (EDTA), Vacutainer, Becton Dickinson) for all animals included in the study, 1 hour before (only on day 1) and 5 hours after anti-CD41 injection.

### Hematology

The blood was homogenized and the cellular composition of blood was determined using a 5-part-differential hematology analyzer (MS9-5, Melet Schloesing Laboratoires).

### Data Analysis

Statistical evaluation of differences between the experimental groups was performed using a one-way ANOVA followed by a Bonferroni post-test (for comparing all pairs of the group). All tests were performed with GraphPad PRISM, version 4.03 for Windows (GraphPad Software Inc., San Diego, Calif., www.graphpad.com).

### Results

Platelet depletion, which reflects the extent of thrombocytopenic purpura, was analyzed with a hematology analyzer.

The data are presented in Table 4 below, in which values corresponding to platelet count are expressed in units of 10³ platelets per μL blood.

| | Mouse | | | | | |
|---|---|---|---|---|---|---|
| Group | number | Day 1 | Day 2 | Day 3 | Day 4 | Day 5 |
| 1 | 1 | 621 | 638 | 618 | 696 | 711 |
| | 2 | 616 | 549 | 648 | 676 | 713 |
| | 3 | 601 | 654 | 760 | 685 | 656 |
| | 4 | 629 | 650 | 609 | 718 | 701 |
| | 5 | 645 | 665 | 652 | 684 | 749 |
| 2 | 6 | 614 | 437 | 248 | 200 | 190 |
| | 7 | 640 | 511 | 142 | 111 | 49 |
| | 8 | 668 | 548 | 392 | 185 | 109 |
| | 9 | 615 | 612 | 310 | 253 | 119 |
| | 10 | 612 | 585 | 190 | 178 | 162 |
| 3 | 16 | 644 | 487 | 357 | 217 | 126 |
| | 17 | 621 | 506 | 277 | 81 | 157 |
| | 18 | 623 | 528 | 340 | 105 | 73 |
| | 19 | 643 | 435 | 353 | 71 | 89 |
| | 20 | 690 | 509 | 331 | 350 | 87 |
| 4 | 21 | 686 | 500 | 455 | 391 | 382 |
| | 22 | 694 | 604 | 531 | 368 | 368 |
| | 23 | 599 | 514 | 432 | 139 | 198 |
| | 24 | 661 | 455 | 466 | 86 | 59 |
| | 25 | 659 | 509 | 255 | 54 | 46 |
| 5 | 26 | 683 | 471 | 391 | 338 | 454 |
| | 27 | 646 | 537 | 368 | 383 | 432 |
| | 28 | 670 | 488 | 420 | 331 | 400 |
| | 29 | 652 | 693 | 394 | 228 | 190 |
| | 30 | 625 | 527 | 521 | 476 | 404 |

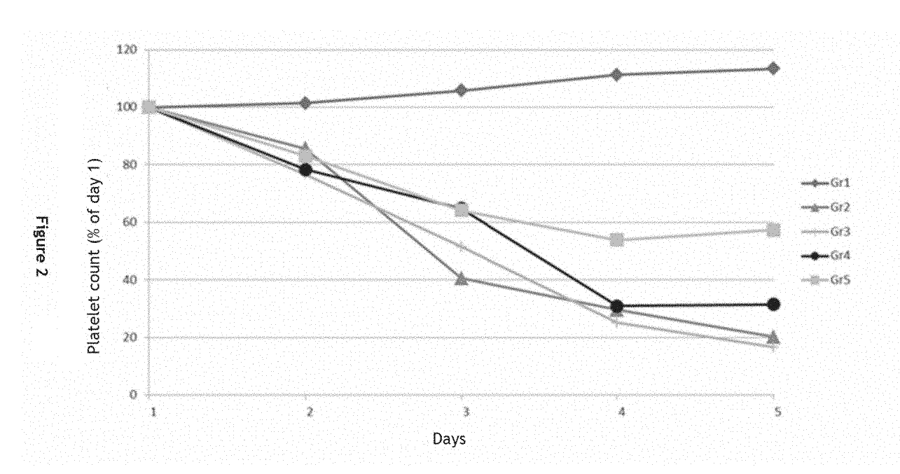

The same data, reported as a percentage of day 1, are represented graphically in FIG. 2.

The data obtained for group 1 (administration of saline solution alone, no administration of platelet-targeting anti-CD41 antibody) and group 2 (administration of platelet-targeting anti-CD41 antibody and treatment with saline solution alone) confirm that the selected model is relevant, as:
administration of saline solution alone has no significant effect on platelet count, and
administration of platelet-targeting anti-CD41 antibody in the absence of effective treatment (saline solution) leads to platelet depletion.

In the negative control group (group 3) injected with anti-CD41 antibodies and treated with erythrocytes alone, the results clearly show no effect of erythrocytes alone on platelet count.

In the positive control group (group 5) injected with anti-CD41 antibodies and treated with erythrocytes and IVIG, the results show, as expected, a significant improvement in platelet count.

In group 4 injected with anti-CD41 antibodies and treated with erythrocytes and anti-A/anti-B, the results show a significant increase in platelet count on day 3, relative to control group 3. On day 3, the results obtained in group 4 having undergone treatment with erythrocytes and anti-A/anti-B are comparable to the results obtained in group 5 having undergone IVIG treatment.

The results show that anti-A and anti-B polyclonal immunoglobulins are effective, in the presence of AB+ erythrocytes, in reducing platelet depletion induced by anti-CD41 antibodies in C57BL/6j mice.

### Conclusions

The data presented above confirm the therapeutic efficacy of anti-A and/or anti-B polyclonal immunoglobulins in the treatment of idiopathic thrombocytopenic purpura (ITP), in a murine model.

### REFERENCES

Aster R H, Bougie D W. Drug-induced immune thrombocytopenia. N Engl J Med. 2007 Aug. 9; 357(6):580-7.
Cines D B, Blanchette V S. Immune thrombocytopenic purpura. N Engl J Med. 2002 Mar. 28; 346(13):995-1008.
Ketton J G, Keystone J, Moore J, Denomme G, Tozman E, Glynn M, Neame P B, Gauldie J, Jensen J. Immune-mediated thrombocytopenia of malaria. J Clin Invest. 1983 April; 71 (4):832-6.
Peterson J A, McFartand J G, Curtis B R, Aster R H. Neonatal alloimmune thrombocytopenia: pathogenesis, diagnosis and management. Br J Haematol. 2013 April; 161(1):3-14.
Thorpe S J, Fox B J, Dolman C D, Thorpe R. Anti-A and anti-B activity in batches of different intravenous immunoglobulin products determined using a direct haemagglutination method. Biologicals. 2005 June; 33(2):111-6.
Thorpe S J, Fox B, Sharp G, Heath A B, Behr-Gross M E, Terao E, Virata-Theimer M L, Yu M W. International collaborative study to evaluate candidate reference reagents to standardize haemagglutination testing for anti-A and anti-B in normal intravenous immunoglobulin products. Vox Sang. 2009 August; 97(2):160-8.
Thorpe S J, Fox B, Sharp G, Heath A B, Behr-Gross M E, Terao E, Virata-Theimer M L, Yu M W. International collaborative study to establish reference preparations to standardise haemagglutination testing for anti-A and anti-B in normal intravenous immunoglobulins by the direct method. Pharmeur Bio Sci Notes. 2010 April; 2010(1):39-50.
WHO/BS/08.2091
Winiarski J. Platelet antigens in varicella associated thrombocytopenia. Arch Dis Child. 1990 January; 65(1):137-9.
WO99/64462
WO01/27623
WO02/092632
WO2007/077365

1. Composition comprising human polyclonal immunoglobulins, characterized in that at least 80% by weight of human polyclonal immunoglobulins present in the composition are human anti-A and/or anti-B polyclonal immunoglobulins, for use as a medicament.

2. Composition comprising human polyclonal immunoglobulins, characterized in that at least 80% by weight of human polyclonal immunoglobulins present in the composition are human anti-A and/or anti-B polyclonal immunoglobulins, for use as a medicament in the treatment of an autoimmune disease and/or a polycythemia.

3. Composition according to claim 2, for use according to claim 2, as a medicament in the treatment of an autoimmune disease.

4. Composition according to claim 3, for use according to claim 3, characterized in that the autoimmune disease is idiopathic thrombocytopenic purpura (ITP).

5. Composition according to claim 2, for use according to claim 2, as a medicament in the treatment of a polycythemia.

6. Composition according to claim 5, for use according to claim 5, characterized in that the polycythemia is primary or secondary polycythemia.

7. Composition according to any one of claims 1 to 6, for use according to any one of claims 1 to 6, characterized in that the medicament is intended for blood group A, B, or AB patients.

8. Composition according to any one of claims 1 to 7, for use according to any one of claims 1 to 7, characterized in that human polyclonal immunoglobulins represent at least 85% by weight of the total proteins of the composition.

9. Composition according to any one of claims 1 to 8, for use according to any one of claims 1 to 8, characterized in that said composition comprises both human anti-A polyclonal immunoglobulins and human anti-B polyclonal immunoglobulins, and in that the weight ratio of human anti-A polyclonal immunoglobulins to human anti-B polyclonal immunoglobulins (anti-A:anti-B) is comprised between 1:10 and 10:1.

10. Composition according to claim 9, for use according to claim 9, characterized in that the weight ratio of human anti-A polyclonal immunoglobulins to human anti-B polyclonal immunoglobulins (anti-A:anti-B) is comprised between 2:1 and 10:1.

11. Composition according to claim 9, for use according to claim 9, characterized in that the weight ratio of human anti-A polyclonal immunoglobulins to human anti-B polyclonal immunoglobulins (anti-A:anti-B) is comprised between 1:10 and 1:2.

12. Composition according to claim 9, for use according to claim 9, characterized in that the weight ratio of human anti-A polyclonal immunoglobulins to human anti-B polyclonal immunoglobulins (anti-A:anti-B) is comprised between 3:10 and 7:10, advantageously between 4:10 and 6:10.

13. Composition according to any one of claims 1 to 12, for use according to any one of claims 1 to 12, characterized in that at least 90% by weight of human polyclonal immunoglobulins present in the composition are IgG.

14. Composition according to claim 13, for use according to claim 13, characterized in that at least 40% by weight of human polyclonal immunoglobulins of IgG isotype present in the composition are subclass IgG2.

15. Composition according to claim 13, for use according to claim 13, characterized in that it has an IgG2:IgG1 weight ratio of at least 0.8.

16. Composition according to any one of claims 1 to 15, for use according to any one of claims 1 to 15, characterized in that it has a human anti-A and/or anti-B polyclonal immunoglobulin concentration superior to 1 g/L.

17. Composition according to any one of claims 1 to 16, for use according to any one of claims 1 to 16, characterized in that the composition according to any one of claims 1 to 16 is administered in combination with another therapeutic agent selected from the drugs useful in the treatment of an autoimmune disease and/or a polycythemia.
